(19) United States
(12) Patent Application Publication
(10) Pub. No.: US 2005/0278798 A1
(43) Pub. Date: Dec. 15, 2005

(54) Method of breeding laboratory animals to optimize tumor suppression and tissue repair functions in vivo and improved methods for conducting experiments and safety testing

(76) Inventor: Bret S. Weinstein, Olympia, WA (US)

Correspondence Address: SHELDON & MAK, INC, 225 SOUTH LAKE AVENUE, 9TH FLOOR, PASADENA, CA, 91101, US

(21) Appl. No.: 11/126,047

(22) Filed: May 9, 2005

Related U.S. Application Data
(63) Continuation of application No. 09/884,183, filed on Jun. 19, 2001.
(60) Provisional application No. 60/212,812, filed on Jun. 19, 2000.

(51) Int. Cl. A01K 67/027

(52) U.S. Cl. 800/18

(57) ABSTRACT

Animals such as mice used in laboratory tests are breed in such a way that the distribution of telomeres yields improved results. This can be accomplished by randomly assigning a breeding period to each animal, by otherwise influencing telomere distribution or by providing multiple populations of animals with different characteristics.

### CROSS REFERENCE TO RELATED APPLICATION

The present application claims priority from U.S. Provisional Patent Application No. 60/212,812, filed Jun. 19, 2000, and from co-pending U.S. patent application Ser. No. 09/884,183, filed Jun. 19, 2001, entitled “Method of Breeding Laboratory Animals to Optimize Tumor Suppression and Tissue Repair Functions in Vivo and Improved Methods for Conducting Experiments and Safety Testing.” The entire disclosure of these documents is hereby incorporated by reference.

### FIELD OF THE INVENTION

The present invention relates to a method for overcoming a problem caused by modern protocols for the breeding of laboratory animals. It further relates to overcoming similar problems that may occur when animals are bred for other purposes such as agriculture and the zoo and pet trades.

### BACKGROUND OF THE INVENTION

The rate of genetic change in response to selection (both under natural and artificial conditions, and specifically in captively bred animals) will be an inverse function of the generation time. The more quickly a species breeds (or is allowed to breed), the faster it will change in response to selection.

In captivity, the timing of breeding for domestic animals, access to resources, as well as exposure to risks and hazards, is controlled (directly or indirectly, intentionally or inadvertently, completely or in part) by humans. This control acts in a fashion analogous to natural selection. This artificial selection will also have the effect of adjusting the rate of aging and the effectiveness of tumor suppression (again, the parameters will tend to shift together, as an inversely-varying pair) so as to maximize fitness in the particular breeding environment. In this way, captive animals can be expected to evolve in response to the selection imposed on them by humans.

The strength of this selective force will also be proportional to the endogenous risk of tumor formation, and the hazard posed by tumors of different sizes and growth rates. Thus, body size is an important variable. Since cell size does not vary significantly with body size, larger animals contain more cells. The likelihood of a tumor being initiated should be proportional to the number of cells from which it might arise. Thus, smaller animals (composed of fewer cells) have a relatively reduced risk of tumor formation. But, smaller animals are more likely to be compromised by a tumor of a given size than are large animals. Hence, once initiated, tumors are more costly and dangerous in small animals.

It is expected that selection on vertebrate species bred in captivity will adjust the effectiveness of tumor suppression and the rate of aging (as an inversely-varying pair of parameters) by modifying the length of a species' telomeres.

Laboratory mice (for example) are placed in a very unusual circumstance by human breeders. They are allowed to breed for eight months (as currently dictated by a National Research Council protocol), then automatically retired. This is equivalent to death from the perspective of selection. The mice which are most successful (fit) are those that start reproducing early and maintain a high rate of reproduction for eight months. Anything that happens to these animals after they are retired is selectively irrelevant.

Mice are small and are thus composed of relatively few cells. Because they have relatively few cells, tumors are relatively unlikely to start. Furthermore, eight months is a short time for a tumor to be initiated by a mutation (or more usually, several mutations in the same cell) and to grow large enough to diminish the reproductive output of a breeder mouse.

The net effect, in lab mice, is that there is minimal selection to maintain tumor-suppression. The selective benefit of tumor-suppression is nearly zero, as breeder mice are no longer allowed to transmit genes to later generations after the breeders are eight months old.

Further, there is powerful selection to slow the aging process in these animals, as their fitness increases with the number of offspring they can generate in the eight month breeding period. Thus, animals which start breeding early and bread at a high rate are most fit.

More than half a century of captive breeding in this environment appears to have radically altered the telomeres of lab mice compared to wild mice (see appended Literature Cited). The telomeres of lab mice are typically ten times longer than the telomeres of normal (wild-type) mice.

As a consequence of their ultra-long telomeres, lab mice

a. Overwhelmingly die of tumors (if allowed to live long enough).

b. Other than tumors, show few signs of decline with age.

c. Have an extraordinary ability to regenerate tissue with age.

d. Have an extraordinary ability to repair damage throughout life.

e. Typically live only two years before they die, generally from tumors (compared to wild mice of the same species, which can live 3-4 years).

That causes a serious problem. When we test drugs, pesticides and other chemical agents or procedures on lab mice, we are almost certain to overestimate the danger of cancer and to underestimate the danger of tissue-damage and accelerated aging. Notice that some substances (e.g. saccharin) that have been shown to cause cancer in lab mice, seem to produce no such effect in humans. Conversely, Seldane (terfenadine), Fen-Phen (fenfluramine, phentermine, dexfenfluramine), and Ritalin (methylphenidate), which are all now suspected of doing tissue damage in humans (generally first evident in the heart) did not cause enough harm to laboratory animals to raise the appropriate questions about their safety.

### SUMMARY OF THE INVENTION

To permit more accurate laboratory tests that are better to predict the number of telomeres of laboratory mice must be controlled or selected in a manner appropriate to the test being conducted.

### PREFERRED EMBODIMENTS

Breeders need to breed mice (and other model organisms) in such a manner that laboratory tests can give researchers an accurate picture of the probable risks, costs, hazards and dangers that humans, pets and other species are likely to face when exposed to the agents and procedures being tested. Breeding them in the traditional way will inevitably produce distortions such as the ultra-long telomere problem that currently plagues modern lab mice. The solutions are as follows:

1. Assign each animal in the breeding population a unique number. Then use a bounded random number (or pseudo-random number) source to assign breeding period start and end dates (the period the animal will be allowed to continue breeding).

2. Define a desirable distribution of ages for breeding initiation and retirement. Divide breeder animals into categories by a non-random, but arbitrary criterion (to prevent assortative effects, such as the creation of sub-populations with distinct telomeric characteristics).

3. Adjusted telomeres adjusted periodically in breeding colonies by modifying the germ-line (sperm and egg cells, or progenitors of same) in some or all individuals, using a combination of methods.

These methods include:
Enzymatic lengthening of telomeres using the enzyme telomerase, or its equivalent, to lengthen telomeres.
Transferring of desirable telomeres, or chromosomes containing desirable telomeres, into target cells which will ultimately produce germ-line cells.
Shortening telomeres via in vitro mitotic cell culture. Chromosomes so altered need to be introduced into germ-lines directly, or indirectly through nuclear transfer which will eventually generate a germ-line.
Cloning, using nuclear transfer, to alter telomere length (which has been demonstrated to be possible in both directions under different protocols). This indirect technique can be used to introduce a desirable distribution of telomere lengths in a breeding population of animals intended for safety testing of chemical agents or procedures (without the need for direct manipulation of telomere lengths).

4. Telomere lengths can be assessed directly in breeder animals, and selection can be applied with respect to which animals are allowed to breed. This technique can produce alterations in the distribution of telomere lengths within the breeding population. Such changes can be produced by restricting or eliminating the fitness of individuals in the breeding pool that have telomere lengths outside of an optimal range. A similar effect can be produced by restricting or eliminating the fitness of individuals whose telomeres, though within the optimal size range, are of a particular size that is more abundant in the population than is desirable.

5. The distribution of telomere lengths in a breeding pool can be modified by introducing individuals from the wild, or from another colony, with telomeres of a desirable length.

6. Telomeres can be modified within a cell or an individual, by triggering as yet undiscovered mechanisms that regulate telomere length (e.g. mechanisms that coordinate telomere lengths between chromosomes).

All of the above methods (1-6) can also be applied with a particular optimal telomere length as a target, or an optimal distribution of telomere lengths as a target.
a. A range (distribution) of lengths is desirable for model organisms to be used for generalized testing. A range of telomere lengths surrounding an optimal balance point (between tissue repair capacity and tumor suppression effectiveness) would allow simultaneous testing for carcinogenic effects as well as tissue damage and accelerated aging effects.
b. A fixed optimum target for telomere length can be set in order to produce model organisms better suited to illuminate dangers of one of two opposing types: carcinogenic effects, and tissue damage/accelerated aging. Testing an agent on two groups (i.e., populations) of model organisms (one optimized to reveal tumor risks, the other optimized to reveal tissue damage risks) will provide better total resolution than testing such an agent on a single population optimized to reveal all risks.

In principle, the problem may be recognized in, and the solution applied to, for example, any vertebrate species in which the age of breeding is controlled, intentionally or incidentally, by humans. The problem is most acute, and the method most useful, in animals with short generation times (inter-birth intervals) and small adult body sizes. Animals for which the method is, in principle, applicable are all vertebrates. All mammals are subject to this method including members of the orders Carnivora (such as Canis lupus, Canis familiaris, Felis silvestris and Felis catus), Primates (such as Pan troglodytes, Pan paniscus, Pongo pygmaeus and Cercopithecus pygerythrus) and Artiodactyla (such as Sus scrofa, Bos taurus, Capra hircus and Ovis aries). Species for which the method will be particularly useful are in the mammalian orders Rodentia (rodents commonly bred for experiments are Mus musculus, Mus domesticus, Mus spretus, Rattus rattus, Rattus norvegicus), Insectivora (moles and shrews) and Lagomorpha (rabbits and hares). Also subject to this analysis and method are innumerable fish, amphibian (e.g., frogs in the genus Xenopus), reptile and bird species.

### DETAILED DESCRIPTION

Telomeres are DNA sequences at the tips of chromosomes. When a cell divides, a small amount of telomere is lost. Thus, the length of an animal's telomeres at birth dictates the total number of progeny cells that any particular cell can produce in a lifetime (the Hayflick limit). When the telomeres of a cell are below a critical threshold, the cell can no longer divide.

Some have argued that the finite capacity to produce offspring cells is a tumor-suppressor mechanism. If a cell gets a mutation that makes it divide indefinitely, the dividing population of cells eventually runs out of telomere. At this point the tumor is arrested and stops growing, generally before it compromises the functioning of vital tissues.

Others have argued that the finite number of descendent cells that individual somatic cells can produce results in failure of bodily tissues over time. As cells are replaced (to compensate for wear and tear) and cell lines reach their Hayflick limits and expire, vertebrates become more vulnerable to physical damage. Thus, as a vertebrate ages, it becomes more susceptible to disease and less able to do useful things, such as run quickly, resist disease, repair physical damage, attract mates, etc.

Not all exhausted cell lines are replaced (compensated). Of those that are replaced, many will be replaced with the progeny of neighboring cells specialized for slightly different tasks. Together, these two processes have the effect of increasing histological (tissue) entropy(disorder). This disorder is a primary cause of bodily decline with age.

We are caught in an evolutionary trade-off between tumor suppression and tissue-repair. If natural selection increases the length of our telomeres, we will age more slowly, but be increasingly prone to get tumors. If selection shrinks our telomeres, we become more resistant to tumors but we will degrade more quickly with age.

This trade-off is inescapable given the basic design of most vertebrates, but the particular rate of aging and the effectiveness of tumor suppression are constantly adjusted (as an inversely-varying pair of parameters) by selection. They are adjusted so as to maximize organismal fitness (the number of copies of an individual's genes that are transmitted into the next generation) given the parameters of the environment in which the lineage is reproducing.

In the wild, natural selection is expected to modify that pair of parameters based on the average likelihood of survival between reproductive opportunities. In a very risky environment in which each day, month, year, etc. carries a large risk of death for an individual, natural selection will adjust the balance in favor of better tumor suppression. The reason for this is that the cost associated with that shift (more rapid senescence) is diminished in such a risky environment since the individual is unlikely to live long enough to suffer the effects of aging.

In a low risk environment, the fitness cost of rapid senescence is potentially great. As such, natural selection should be expected to retard the aging process at a significant cost (namely, the increased risk of tumor formation throughout the lifespan). Organisms with long, active lives inevitably sustain tissue damage. If they are composed of functionally redundant modules, then continued viability does not depend on tissue repair. But, in vertebrates, life-long maintenance and repair of vital tissues is necessary. Due to the stochastic nature of damage, repair must be locally self-activating, so that the destruction of tissue is sufficient to initiate replacement by adjacent cells. However, local self-activation is a liability, because mutations which disable a cell's capacity to sense healthy neighbors will tend to trigger runaway proliferation. This propensity to form tumors is an inherent obstacle to the evolution of large, long-lived, self-repairing organisms. Mechanisms which allow for extensive tissue repair while inhibiting the frequent production of tumors should be regarded as major evolutionary innovations—prerequisites to the evolution of life history strategies like those of most vertebrates. The nature of that innovation in vertebrates appears to be a soma-wide fail-safe mechanism which reins in runaway cell lines, but unavoidably results in the progressive degradation of tissue structure and function with age. It may seem we are a taxon plagued by senescence and tumors, but in fact we are the beneficiaries of extensive, vigorous lives that result from selection's remarkable efficiency at simultaneously minimizing the harm of these two opposing hazards.

Why do we get tumors and why do we grow old? These questions are pieces of a larger puzzle: how is it possible for a highly differentiated, self-repairing organism composed of millions, billions or trillions of cells to live long enough, in a mutagenic environment, to reproduce, without a single cell escaping the normal developmental program and becoming a deadly tumor? An answer is provided by synthesizing knowledge from two distinct approaches to vertebrate senescence: evolutionary theory and experimental gerontology.

Historically, these approaches have been practiced almost independently. Evolutionists have remained largely unconcerned with the proximate mechanisms of aging and gerontologists have been lax about the ultimate explanations which underlie their discipline. But comprehension of genetic and cellular machinery has now progressed to the point that evolutionary theory and empirical findings have begun to mirror each other. Not only can these explanations for aging now be profitably synthesized, but that synthesis can accelerate progress in both parent disciplines. Evolutionists and gerontologists need a common body of theory and knowledge as well as a language which allows for meaningful discourse.

I. Synthesizing Two Views of the Aging Process

Senescence: The Evolutionary Approach

All else being equal, longer lives provide more reproductive opportunities than shorter lives, so natural selection must oppose senescence. Compared to the immense challenge of building a self-assembling, ten trillion cell organism (such as a human), the maintenance of such an organism should be relatively simple. Yet selection has failed to eliminate senescence from any vertebrate.

Elaborating on Medawar (1952), Williams (1957) explained the persistence of senescence as follows: Even in the absence of senescence, all lives would be finite because every organism would ultimately die from accident, starvation, predation or disease. Since an organism is always at risk of death, natural selection should favor early reproductive opportunities over the potential for latter ones. The force of natural selection must, therefore, be strongest at the typical age of commencement of reproduction, when reproductive potential is greatest, and it must decline from that point forward. Therefore, traits which have beneficial effects early in life will tend to spread, even if they are inseparably coupled with deleterious effects that manifest later in life. Individuals are thus endowed with youthful vigor, at the cost of inevitable senescence.

According to Williams, selection modifies a species' rate of senescence (in response to the distribution of mortality risks across the reproductive life-span) by adjusting pleiotropic balances between longevity and youthful vigor. This analysis accounts for a whole spectrum of senescence modes from the sudden expiration of salmon to the very gradual decline of giant land turtles. A giant tortoise, housed in a protective shell, on a remote island, faces very few hazards between reproductive opportunities. Therefore, selection for longevity is strong compared to selection for youthful vigor, producing a very gradual rate of senescence. Conversely, a spawning salmon would face extreme hazards returning to sea and later attempting a second journey upstream. The very low probability of future reproductive opportunities results in a semelparous life-history strategy in which all resources that can be mobilized are invested in progeny rather than maintenance.

Selection could not produce such life-history refinements if it did not retain substantial power during the process of senescence. Though the force of natural selection declines from the onset of reproduction, it descends from a great height and remains strong throughout the reproductive lifespan, even as the effects of senescence are becoming increasingly evident.

This is a critical point, yet it is persistently misunderstood outside of evolutionary biology. In gerontology it is commonly asserted that senescence results from “unselected” late effects of genes (e.g. Harley, 1997; Campisi, 1997a,b). The declining force of selection does eventually approach zero, but that fact is insufficient to explain senescence, because senescence begins at the onset of reproduction, when selection is strongest (Williams, 1957).

Throughout the period of reproduction and offspring-rearing, selection acts to minimize deleterious effects associated with beneficial genes. If we mistakenly believe that senescence is the product of “unselected” costs, then we may harbor great hopes for therapeutic reduction of these effects. Conversely, if we view senescence as the unavoidable costs which remain after selection has acted to minimize harmful effects, then we will correctly view senescence as the same daunting challenge for medical science that it has apparently been for natural selection.

Modifying the Theory of Antagonistic Pleiotropy

Williams' theory has withstood the test of time, and the last four decades have provided few additional theoretical insights. However Williams' treatment is dated in one significant sense. In 1957, little was known about gene expression and regulation. That gap led to an unnecessary reliance on pleiotropy as the inextricable link between early and late genetic effects.

The fact that good and bad effects maybe derived from the same pleiotropic gene does not necessarily imply that those effects are inseparable. If the effects of a gene become deleterious at some point in the life-cycle, the gene can generally be turned off at that point. Only if such regulation is impossible, or early expression itself produces deleterious late effects, is pleiotropy sufficient to explain senescence.

The term ‘pleiotropy’ also needlessly restricts consideration to multiple traits produced by single genes. Williams' logic should apply to any trait with early benefits intrinsically tied to late costs. The costs and benefits need not derive from multiple traits, they may result from a single trait for which the cost/benefit ratio increases with age (Alexander, 1987). Furthermore, the trait need not result from an individual gene; the combined effects of multiple genes may produce emergent costs and benefits.

Consider this example (unrelated to senescence) of a multi-gene trait with inseparable good and bad effects. Animals with a large surface to volume ratio radiate heat efficiently. In many habitats the efficient radiation of heat is beneficial in the summer and hazardous in the winter. Divergent consequences result from this single trait (body shape) produced by multiple genes. Though the trait remains static, the cost/benefit ratio oscillates. The trait spreads in habitats where the net effect is positive.

The logic of Williams' theory applies to any trait or system with early benefits and delayed costs, regardless of the number of genes involved or the number of traits produced. It is both necessary and sufficient that the cost/benefit ratio of the trait or system is initially less than one, becoming greater than at some point after the onset of reproduction.

Telomeres and Senescence: The Experimental Approach

In 1961, Hayflick and Moorhead made an important breakthrough in the experimental study of senescence. They disproved the notion that normal vertebrate cells could divide an indefinite number of times in vitro. They showed that normal somatic cell lines were limited in the number of population doublings they could undergo before growth slowed dramatically, then ceased. Later studies showed that the number of cell divisions occurring before the ‘Hayflick limit’ covaries (between taxa) with lifespan (Hayflick, 1973; Hayflick, 1991; Rohme, 1981) and decreases in humans with age (Allsopp et al., 1992). For many years these findings lacked a mechanistic explanation, but a front-runner has now emerged (Chiu and Harley, 1997; see also de Lange, 1998). DNA polymerase is unable to fully duplicate the tips of chromosomes, resulting in the loss of a small amount of DNA with each successive cell division (Olovnikov, 1973; Watson, 1972). This progressive erosion would be catastrophic if important genes were located at the ends of chromosomes. But the ends of eukaryotic chromosomes consist of long, non-coding, repetitive sequences known as telomeres (Moyzis et al., 1988; Blackburn, 1991). Telomere loss may explain the mortality of somatic cell lines: the erosion of telomeres below a critical length appears to trigger the shutdown of replicative machinery (Griffith et al., 1999).

Clearly, there must also be some means of adding telomeric DNA to chromosome ends. Otherwise germ-lines would be mortal as well. The reverse transcriptase ‘telomerase’ elongates telomeres (Blackburn, 1991; 1992; Feng et al., 1995; Weinrich et al., 1997). Telomerase is active in gametogenesis and undetectable in the vast majority of adult somatic tissues (Kim et al., 1994).

Several lines of evidence support the telomere erosion hypothesis for Hayflick limits.
Telomere length diminishes with cell-line age in vitro and in vivo (Hastie et al., 1990; Lindsey et al., 1991; Harley et al., 1990).
A remarkably diverse array of immortal somatic cell lines (tumors, which lack Hayflick limits ) express telomerase (Counter et al., 1994; Kim et al., 1994).
Somatic tissues from patients with Hutchinson-Gilford (H-G) and Werner's syndromes (diseases of dramatically accelerated aging) have reduced proliferative capacities in vitro. H-G patients have short telomeres at birth (Allsopp et al., 1992). Werner's patients experience rapid erosion of initially normal telomeres (Faragher et al., 1993).

The association of aberrant telomeres with apparently accelerated aging suggests that Hayflick limits may explain more than just the mortality of individual cell lines. The limited proliferative capacity of somatic cells may underlie a general mechanism of body-wide senescence.

This possibility led to an experiment, with equivocal results (Rudolph et al., 1999). A strain of laboratory mice with two disabled copies of a gene necessary for telomerase activity was produced (Blasco et al., 1997). This telomerase-negative strain did exhibit accelerated aging, but only after six generations. Even then, the effect was not uniform. Mice in the sixth generation seemed to senesce prematurely in some tissues and not others. These results strengthened the argument that telomeric erosion is involved in somatic senescence, but suggested that the role of telomeres in the phenomenon of senescence might be limited to those few somatic tissues with high endogenous rates of turnover (Lee et al., 1998). The six generation delay was taken to imply that normal senescence, of the type that occurs in a single generation, must involve important undiscovered factors (Rudolph et al., 1999). We will reinterpret these results below.

Telomeres and Cancer

The connection among telomeres, Hayflick limits, and the phenomenon of senescence is important whether telomeres are the primary mechanism or just one of several. But, telomere regulation has significance beyond the issue of our gradual decline with age. Telomere regulation appears central to another great enemy of the old: cancer.

Activation of telomerase appears to be a necessary step in most transformations of normal tissue into tumors (Kim et al., 1994; Feng et al., 1995). The connection of cancer and senescence to the same mechanism is not serendipity, it is a window into a remarkable adaptation (produced by antagonistic selection), the balance of which we may find difficult to improve.

The Synthetic Approach: Hayflick Limits and Antagonistic Selection

Juxtaposing an evolutionary perspective on senescence, with the gerontological and oncological view of telomeres, it appears that Hayflick limits evolved as tumor suppressors that rein in runaway cell lines, but that these same limits preclude indefinite maintenance of the soma, causing gradual degradation of function. It seems the telomere/telomerase system is analogous to an antagonistic pleiotropy of the type Williams (1957) predicted.

Kipling (1995) briefly proposed a similar interpretation (without reference to Williams' theory), but there has been no apparent discussion of his idea or its implications. Others (Harley, 1997; Wright et al., 1996) have mentioned antagonistic pleiotropy in this context, but have evidently failed to appreciate the advances made by Williams (1957) over Medawar (1952).

The declining force of natural selection with age is not sufficient to explain senescence during prime reproductive years. Only when senescence is recognized as a selective consequence of inherent trade-offs can we fully understand the nature of aging.

The Reserve Capacity Hypothesis

A new term facilitates discussion of vertebrate telomeres. We will use ‘reserve capacity’ to refer to the remaining in vivo proliferative potential of a cell or cell line.

The relationship of Hayflick limits to tumors seems relatively straightforward. When a cell is damaged such that it begins to over-proliferate, it ultimately reaches its Hayflick limit and proliferation ceases. The greater the reserve capacity of the progenitor cell, the larger the resultant mass of growth-arrested daughter cells will be. We regard this mass of cells as a ‘proto-tumor’, possessing the first of several mutations necessary for tumorigenesis and cancer.

Because cells will tend to have more reserve capacity early in an organism's life, younger individuals should tend to produce larger proto-tumors than older individuals. Since each cell in a proto-tumor presents an equivalent opportunity for the acquisition of telomerase activators, we predict that proto-tumors produced early in life carry proportionately higher risk of becoming mature tumors than proto-tumors generated late in life. This effect will be exacerbated by the fact that proto-tumors formed at an early age will tend to have more time in which to accumulate further genetic changes. The risk from any particular proto-tumor should diminish with time, as growth-arrested cells are lost by normal cellular attrition. Risk reduction may be accelerated by programmed apoptosis.

Somatic Senescence Due to Cell Line Attrition and Increasing Histological Entropy

To our knowledge, no explicit mechanism linking Hayflick limits to the phenomenon of vertebrate aging has been proposed. We offer the following first approximation.

Development continually increases histological differentiation and specialization, which are maximal when an organism becomes a reproductively capable adult. Throughout life, damage and regular cellular turnover result in cells being lost from the soma and replaced. Some cell lines will exhaust their reserve capacity; when these exhausted cells are eventually lost, they will be replaced by neighboring cell lines, if they are replaced at all.

We propose that the uncompensated loss of some cell lines, coupled with the replacement of others by neighboring cell lines adapted to slightly different roles, diminishes histological organization. By our model, body-wide senescence results from the combined effect of uncompensated cellular attrition and increasing histological entropy, both of which will diminish an organism's efficiency at accomplishing whatever tasks differentiation initially evolved to address. Senescence of this type should progress at a non-linear rate, accelerating with age as fewer cell lines maintain and repair an ever larger proportion of the body.

The aging of human skin appears to progress as our model predicts. Skin thickness decreases approximately 25% between the fourth and eighth decade of life (Black, 1969), and entropy increases:
“The epidermis of older individuals exhibits a marked variation in thickness (often in the same histologic section) and a disparity in the size, shape and staining quality of the basal cell nuclei under light microscopy. There is also a loss of the orderly alignment of cells along the basement membrane and a disruption of the gradual upward uniform differentiation present in the epidermis of younger individuals . . . Electron microscopic studies show that the basal cells of the flattened epidermis of old individuals lack villi.” (Balin, 1994, p. 348) “Deletion and derangement of small blood vessels is found in aged skin, with sun-damaged skin . . . being the most severely affected.” (Balin, 1994, p. 364)

Cardiovascular disease may provide an example of the consequences of cell line attrition and increasing histological entropy. Cells in portions of the vascular system that sustain relatively high levels of wear and tear have short telomeres, implying a history of cellular replacement (Chang and Harley, 1995) and likely cell line attrition. These areas fail to produce a protective layer of cells characteristic of younger tissue, and consequently have an increased propensity to develop artherosclerotic plaques (Chang and Harley, 1995).

One Source, Three Sinks

Vertebrates use reserve capacity in growth, maintenance, and repair; each process erodes telomeres, reducing proliferative potential. Though antagonistic pleiotropy and accumulated damage hypotheses have traditionally been viewed as alternative explanations for senescence, the finite reserve capacity approach integrates them. Damage, even if it is functionally fully repaired, will accelerate the aging of the tissue in question by limiting its future capacity for maintenance and repair. The liver of a heavy drinker, for instance, may function essentially as well at 40 as it did at 25, but should fail more rapidly than the liver of a non-drinker, even if the destructive behavior ends before damage is evident. Any factor which damages tissue, including somatic mutations, pathogens, mechanical wear or trauma, oxidative stress and free radicals, will promote a local increase in that tissue's rate of senescence.

Selection should tend to optimize reserve capacity based on a species' timing of reproduction and the typical rate of cellular repair and turnover. Although telomere erosion begins at whatever point in ontogeny telomerase is inactivated in the soma, selection should adjust reserve capacity so the loss of cell lines does not begin before the usual age of first reproduction. Selection should further act to coordinate reserve capacities among tissues so that senescence is synchronized throughout the body (as per Williams, 1957).

But, because of the stochastic nature of environmental insults, past selection cannot predict the reserve capacity needs of individual organisms nor the organs on which they depend. An otherwise healthy individual may die from the premature senescence of a particular tissue which has had an unusual history of damage. Likewise, individuals are exposed to unpredictable differences in rates of damage. We should therefore expect dissynchrony of senescence rates between conspecific individuals, and between organs within an individual, despite the synchronizing force of selection.

Selection can adjust telomere lengths based on species' average body size, rates of damage and mortality risks, but selection based on averages will not produce ideal telomere lengths for individuals. How could selection optimize the telomere length on a chromosome passed from a 5′6″ father to his 6′1″ son? This obstacle may explain why the positive interspecific correlation between body size and longevity (explained in Williams, 1957) is reversed within species. For example, even when the effects of obesity are controlled for, larger humans (Samaras and Elrick, 1999; Samaras and Storms, 1992) and dogs (Michell, 1999; Li et al., 1996) tend to be comparatively short lived. The extra cell divisions required to become larger, by diminishing reserve capacity at maturity, may shorten life-span by reducing the capacity of larger individuals to maintain and repair their tissues. We expect smaller individuals to suffer a greater per cell risk of developing tumors due to longer-than-optimal telomeres at maturity. At the same time they should show an increased resistance to other senescent effects. Since smaller individuals are composed of fewer cells, we do not expect their increased per-cell tumor risk to fully counteract their decreased rate of senescence. Therefore, within a species, smaller individuals should tend to live longer.

Reinterpreting Experimental Results

Senescent Cellular Phenotypes: Misregulation or Adaptive Response?

Upon reaching a Hayflick limit, many cell types begin expressing genes that were previously untranscribed, and cease expression of previously active genes (Smith and Pereira-Smith, 1996). Several workers have conjectured that somatic senescence of individuals results from the progressive accumulation of cells with “senescent phenotypes” (Campisi, Dimri and Hara, 1996; Johnson, Sinclair and Guarente, 1999; Ly et al., 2000). To our knowledge no one has proposed a mechanistic connection between these phenotypes and organismal aging. Instead, the phenotypic changes are asserted to result from “misregulation”. The implicit assumption is that expired cell lines accumulate late enough in life that selection lacks the power to regulate their function. We propose a contrary interpretation.

Williams (1957) argued that late negative effects would spread if pleiotropically associated with early benefits. He went on to argue that selection would then produce modifiers which would minimize the harm caused by these late effects. We suggest that senescent cellular phenotypes are adaptations which limit the harm caused by cell line expiration.

The misregulation hypothesis is apparently falsified by the very data used to support it. Ly et al. (2000) compared gene expression amongst people from three age classes and children with H-G progeria. They found that 50% of the genes whose expression is altered in aging (both accelerated and normal) belonged to two classes, mitosis initiation and progression genes (e.g. spindle formation) and extracellular matrix (ECM) modification genes. If transcriptional changes were the result of misregulation then we should expect a random pattern of modification reflecting a lack of stabilizing selection on gene regulation. Instead regulatory changes were observed in groups of functionally related genes, suggesting the activity of selection.

The direction of the regulatory changes are equally suggestive of selection. Mitosis-related genes were downregulated with age. This is unsurprising as senescent cells, which do not divide, are unlikely to need spindles or other mitotic machinery.

In contrast to the uniform down-regulation of mitosis-related genes, some of the genes which modify the ECM were upregulated and others downregulated. Downregulated genes were associated with construction of the ECM while upregulated genes were associated with its disassembly. This is consistent with earlier findings which suggest that senescent cells decrease the production of collagen and increase production of collagenase, an enzyme which breaks down collagen and thereby facilitates the remodeling of the ECM (Campisi, 1997a).

We propose that selection has constructed a system that locally breaks down the ECM as cells are nearing their Hayflick limits, thereby facilitating cellular replacement. Early in life, the ECM maintains the developmentally optimal placement of cells. But this is an impediment to cell motility. As cell lines become unable to replace themselves, adjacent cell lines may not be able to fill vacated spaces with the ECM in place. Selection may have programmed senescent cells to locally dismantle the ECM, paving the way for their eventual replacement by adjacent cell lines. Such modification may itself be part of a tradeoff. The breakdown of the ECM likely facilitates the movement of metastatic cancer cells. The degree of breakdown of the ECM may reflect a balance between the beneficial replacement of expired cell lines and an increased danger of metastasis.

Lab Mice and Cloned Sheep: Life on Strange Islands

If individuals disperse from a high risk environment to a low risk environment (e.g. a remote island) selection for greater longevity should slow the rate of senescence (Williams, 1957). The evidence that selection does this in the wild is strong (Austad, 1993; Ricklefs, 1998; Reznick, 1997). We expect that selection adjusts telomere lengths to postpone senescence under such circumstances. This adjustment must come at some cost, such as increased risk of tumors and/or an increased burden from larger proto-tumors.

In the early part of this century, a small number of Mus musculus dispersed into a novel environment: the laboratory. In breeding colonies there is no predation, no resource limitation and spread of pathogens and contaminants is controlled. Perhaps most importantly, breeders are retired at eight months (National Research Council, 1981) so the mice that contribute most to future generations are those that begin reproduction early, and sustain a high rate of reproduction until the cutoff age. The laboratory is remarkably unlike the environment mice originally evolved to exploit, and likely favors a different pattern of senescence.

The telomere systems of laboratory mice are hard to reconcile with the notion of Hayflick limits as tumor suppressors or as the cause of senescence. Compared to humans, lab mice have ‘ultra-long’ telomeres, exceeding human telomeres by an order of magnitude (Kipling and Cooke, 1990). Further, somatic tissues of lab mice produce telomerase, and “spontaneously immortalize” in vitro.

We predicted that the long telomeres observed in laboratory mice would be atypical for mice in general. Greider's lab tested this with a survey of telomere lengths in six species of laboratory mice with shorter histories of captivity than laboratory Mus musculus, and all six had telomere lengths approximately one tenth of those in common lab mice (C. Greider, pers. com.).

The unusual telomere system of lab mice may be an unintended consequence of captive breeding. Retirement of breeders after eight months eliminates selection on late life effects. Tumor-forming mutations take time to occur, and the likelihood of tumor formation is a function of the number of cells in the body, so in mice, tumors may be rare in the first eight months of life, even without the telomeric fail-safe. Further, selection for sustained high reproductive output before eight months should tend to eliminate any cellular senescence occurring before that deadline. Selection acting to simultaneously increase early reproductive output and eliminate senescent effects may elongate telomeres. Because of the inextricable connection between tumor suppression and somatic maintenance, telomere elongation should dramatically increase the risk of tumor formation, but any effects occurring after the breeding cut-off will be selectively irrelevant. Selection for early high rates of reproduction in the absence of selection for longevity should result in a strong propensity for these mice to eventually die from tumors. At all ages, lab mice should be more likely to die of tumors than wild mice raised in similar environments. Lab mice should also be unusually resilient to somatic damage and show few signs of aging other than tumor formation. Alexander (1966, p. 272) presents evidence consistent with this pattern:
“The most striking fact is that even very old [lab] mice (e.g., more than two and one-half years) when killed while still fit have remarkably few pathologies and are almost indistinguishable from young animals.”

Unfortunately, it has been widely assumed and asserted that unusually long telomeres are characteristic of “mice” or even “rodents” in general. The stark differences between lab mice and humans led de Lange (1998) to argue:
“ . . . it seems very unlikely that mice use telomeres as a tumor suppressor system and perhaps with good reason. Since the telomere barrier to proliferation does not manifest itself until many cell divisions have passed, this mechanism may not be useful for a small animal in which a 2 cm mass of misplaced cells could be life-threatening. ”

We agree that the telomere system of small animals would need to arrest very small growths to serve as a useful tumor suppressor, but the conjecture that “mice” don't use this system is premature. The tissues of wild mice might have very limited reserve capacities, thus protecting them from lethal growths, but limiting their life spans.

It is unfortunate that laboratory strains were used to create the telomerase-negative mice. If the experiment had been conducted using mice recently derived from the wild, accelerated senescence might have been observed in the first generation. But even in such an experiment we predict the acceleration of gross senescent effects would be limited to high-turnover tissues because other tissues, which typically use reserve capacity to repair damage, will senesce minimally in the protected environment of the lab.

The unique state of lab mice may lead to erroneous conclusions about tumorigenesis. For example, based on evidence from mice with ultra-long telomeres, Kipling (1997) speculates that “ . . . telomerase expression in mouse tumorigenesis is an innocent bystander rather than a necessary event.” Clearly, telomerase activity, telomere length regulation and spontaneous immortalization must be investigated in newly domesticated mice to separate experimental artifacts from natural phenomena.

Care must also be taken in interpreting the pattern of aging in animals produced through nuclear transfer cloning, such as the sheep Dolly. The nucleus that was used to produce Dolly was taken from an adult sheep (Campbell et al., 1996), and thus had shorter telomeres than a normal sheep zygote, though as yet Dolly does not appear to be senescing abnormally (Shiels et al., 1999). Like lab mice, Dolly lives in a controlled environment, protected from the traumas, illnesses and impurities of a wild or even a typical farm habitat. We expect Dolly to senesce earliest in tissues with high endogenous turnover rates (because her need for damage repair is likely to be minimal), and to display early senescence compared to sexually produced controls reared in the same protected environment. But compared to normal sheep, her senescence may not appear accelerated, as it is likely being slowed by the near absence of environmental insults.

Retarding Senescence with Caloric Restriction: Natural Phenomenon or Laboratory Artifact?

Caloric restriction (CR) is the only experimental treatment shown to dramatically increase longevity in vertebrates. Laboratory mice and rats placed on a restricted diet live significantly longer than controls (Ross, 1976). This has been interpreted as evidence that resource limitation slows the process of senescence. But if, as we suggest, these animals have been selected to not senesce, then slowing senescence should have little effect on their longevity.

We have argued that laboratory mice should overwhelmingly die of tumors. CR may increase longevity of these animals by reducing the risk of tumor formation. CR animals are approximately ⅓ smaller (Sprott and Austad, 1996) and exhibit slower cell replication (Wolf et al., 1995) than controls. By stunting growth (reducing the number of cells in the organism), and by reducing the rate of cell division, CR may simply reduce the likelihood of tumorigenesis.

Such positive effects might also occur in CR vertebrates with wild-type telomeres. Further, by reducing body size and possibly slowing cellular turnover, CR should postpone the exhaustion of reserve capacity. But CR, like famine, will likely have severe negative effects as well, interfering with normal homeostasis and repair. We predict that CR will not dramatically increase longevity in vertebrates with normal telomeric tumor suppressors.

II. Selective Inactivation of the Telomeric Tumor Suppressor

The Counterintuitive Nature of Early Development

If finite reserve capacity is an evolved fail-safe against runaway cell lines, we must consider those times and places where selection has disabled this mechanism. Telomerase is present in the somatic tissues of embryonic placental mammals, but activity ceases before birth (Wright et al., 1996; Ulaner and Giudice, 1997; Yashima et al., 1998). To illustrate why selection responds differently to telomere erosion in early versus late development, we will compare the distinct developmental profiles of two processes: cellular population doubling and resource investment.

In the absence of telomerase, telomere loss is a function of the number of cell population doublings. Division of a zygote into two cells would reduce the mature body's reserve capacity as much as the growth of a 5 trillion cell child into a 10 trillion cell adult (if all cells made an equivalent contribution to growth). The vast majority of cellular doublings occur in early development when the absolute number of cells is very small and the embryo is tiny compared to the adult it will become. In contrast, parental investment of resources is lowest in early development, when most cellular doublings occur, and investment grows with the embryo's size. Because of this asymmetry, the resources placed at risk by early fetal telomerase activity are minimal.

Spontaneous abortions are common in early fetal development, ending approximately 50% of human pregnancies (Stabile, Grudzinskas and Chard, 1992). Early spontaneous abortions are not without cost. In many species breeding periods are narrowly timed and an aborted pregnancy may eliminate a female's reproductive output for the year. The cost of early spontaneous abortions has apparently resulted in mechanisms that reduce such risks, such as maternal aversion to complex foods during early stages of pregnancy. This may function to protect the embryo/fetus from mutagens during an especially vulnerable period (Profet, 1988). We propose that isolating the fetus from mutagens is particularly important while telomerase is active, when runaway cell lines necessarily result in abortion.

In humans the majority of cell divisions occur before the end of the fifth month of gestation, while telomerase is maintaining telomere lengths. The period of telomere maintenance ends, on a tissue-by-tissue basis, beginning in the fourth month and continuing through the fifth month (Wright et al., 1996; Ulaner and Giudice, 1997; Yashima et al., 1998). After this point the fetus begins to accrue resources in the form of body fat. In contrast to the rate of cell addition, which peaks in the fifth month then drops precipitously (Thompson, 1943), the great majority of prenatal weight gain occurs in the later, telomerase-negative period. We interpret this developmental pattern as a mechanism by which selection has minimized the resources placed at risk by developmental telomerase activity.

Though early telomerase activity carries risks, a lack of telomerase during the period of rapid cellular doublings would result in a substantial erosion of the telomeres, accelerating the onset and rate of senescence later in life. It seems selection could solve this problem by lengthening germline telomeres, thus adding reserve capacity to the organism as a whole. Because selection has favored telomerase activity (and its associated risks) over a simple lengthening of telomeres, we expect fetal telomerase activity also provides a significant benefit.

The nature of that benefit may relate to Williams' (1957) argument that selection should tend to synchronize senescence across the soma. If finite proliferative capacities determine the senescence rates of different tissues, and if those rates are to be synchronized by selection, telomere lengths must be adjusted according to the typical rates of cellular turnover of different parts of the soma. Simply lengthening germline telomeres could not produce this synchronization. If telomerase were never active in the soma, the reserve capacity of a particular tissue would simply be an inverse function of the total number of cell divisions that produced it from the zygote. In contrast, tissue-specific telomerase activity can establish inter-tissue synchronization. This could be accomplished at any point in the lifecycle, but it is least costly in early development when (1) the investment placed at risk is minimal, (2) the fetus is insulated from most environmental mutagens, and (3) the number of potential runaway cells is relatively small.

The reserve capacity of mature tissues can be set by adjusting the number of cells in each tissue before telomere maintenance ceases. If it is determined that organ senescence is developmentally synchronized, this will firmly establish that patterns of senescence are products of natural selection, not incidental effects that occur in the absence of selection.

After somatic telomerase is shut down, growth via cell division will reduce tissue reserve capacity. Wistar rats that were growth-retarded prenatally (i.e. during telomere maintenance), but grew to normal size after birth, had shorter telomeres in their kidneys and shorter life spans than control rats (Jennings et al., 1999). Among humans, women that were short at birth but grew to average or above average height had an increased risk of death from coronary heart disease (Forsén et al., 1999). A similar pattern appears to exist in men (Eriksson et al., 1999), though it is confounded by mortality risks associated with obesity rather than “catch-up growth”.

Cellular Over-Proliferation in Early and Late Life: Tumors of Two Natures

If the shortening of telomeres is part of an adaptive tumor suppressor mechanism, why are tumors most common late in life, when telomeres are shortest? Tumors may be divided into two classes: (1) early-life tumors, which arise if telomere lengths are exceedingly long or are maintained by telomerase; and (2) tumors occurring late in life, after telomeres have become critically short. Reserve capacity limitation appears to counter early-life tumors so successfully that we may fail to realize that a serious threat of early-life tumors would otherwise exist. The few systems in which telomere lengths are maintained provide a window into life without the telomere fail-safe.

Childhood Leukemia and Lymphoma

Most of the tumors common in the elderly are essentially unknown in children and young adults. The most common childhood tumors, leukemias and lymphomas, involve hyper-proliferative leukocytes (B- and T-cells) or their progenitors. Leukocytes are responsible for our specific immune response which “learns” to recognize pathogens. When a leukocyte is activated by a matching antigen the cell proliferates, creating a population of cells with variations of the progenitor cell's receptor formula. Iterated clonal selection allows the system to hone in on unfamiliar pathogens and to track antigenic changes in an ongoing infection (Burnet, 1957).

Although most leukocytes will never be stimulated, the subset that become activated must retain an indefinite ability to proliferate. Otherwise pathogens for which immune cells have initially weak affinities would remain elusive, and rapidly changing pathogens could exhaust the proliferative potential of the immune system. Instead, leukocytes produce telomerase upon antigenic activation, allowing for extensive proliferation (Weng, Granger and Hodes, 1997; Buchkovich and Greider, 1996). We suspect that telomerase activity, which is necessary to the functioning of the immune response, is causally linked with the substantial childhood risk of developing leukemias and lymnphomas.

Germline Tumors

Testicular cancer is essentially absent in boys, but beginning at puberty (when gametogenesis begins) the incidence of testicular germ cell tumors jumps, peaking between the ages of 20 and 34 (Horwich, 1996). Germline tissue does not senesce, so spermatogenic cells must maintain their telomeres throughout life, despite undergoing very high rates of cellular proliferation. Spermatogenic cells produce telomerase (Kim et al., 1994), which may explain the occurrence of testicular cancer in young men. In female mammals gametogenesis occurs before birth, so there is no increase in risk of germ cell tumors with puberty. Indeed, minimization of the fitness costs associated with germline tumors may account for the evolutionary shift of female gametogenesis to prenatal development.

Tumors Late in Life

Late-life tumors can arise by at least two pathways. A mutant cell which has become developmentally insensitive to signals halting growth may gain a mutation that activates telomerase. This is statistically unlikely in any individual cell, but since the many cells in a proto-tumor will all carry the initial mutation(s), the risk that one will also gain an additional mutation increases with the number of proto-tumor cells.

The second pathway does not depend on telomerase or a population of cells at increased risk. Typically cells cease proliferation when telomeres become critically short. But a cell carrying a mutation that prevents such arrest may continue to divide and erode the telomere below the threshold necessary to stabilize the chromosome ends. When that occurs, chromosomes become unstable and fuse into closed structures (Greider, 1999). Such chromosome instability has dramatic, unpredictable effects and may lead to excessive growth even in the absence of telomerase. For example, the erratic telomere shortening and resultant chromosomal aberrations characteristic of Wemer's syndrome results in both tumorigenesis and accelerated senescence.

A Senescence ‘Rescue’ Mechanism: Reactivation of Telomerase in Failing Tissues

Telomerase is believed to be inactive in nearly all healthy somatic tissues of adults, but we suspect this is a significant oversimplification. Selection should balance the risk posed by early senescence of a disproportionately damaged tissue, against the risk of tumorigenesis. If relatively early senescence of a tissue threatens the survival of the individual, local activation of telomerase may be a worthy risk. If the exhaustion of cellular reserve capacities was not due to hyper-proliferation, then telomerase will extend the life of the tissue. We predict the existence of such a ‘rescue’ mechanism. However, if the rescued section includes a proto-tumor, tumorigenesis may result. We expect localized activation of telomerase to increase with age and only a subset of such activation to be associated with tumors.

Lanza et al. (2000) found that calves cloned from senescent fibroblasts were born with unusually long telomeres. The use of senescent cells may have inadvertently triggered the rescue mechanism, producing this anomalous result. In any case, we predict that the cloned calves with long telomeres will have increased cancer rates compared to sexually produced calves raised in a similar environment, and will otherwise exhibit relatively delayed senescence.

The failure of telomerase reactivation may be relevant to Hutchinson-Gilford syndrome. H-G progeria is a homozygous recessive condition (Khalifa, 1989; Maciel, 1988) which we predict results from two inactive copies of a gene necessary for telomerase functionality. Without telomerase, the erosion of telomeres during early development would be substantial, and could account for the abnormal ontogeny and early onset of senescence in H-G patients. The inability to rescue senescent tissues by selectively reactivating telomerase may account for the rapid decline of H-G patients compared to normal elderly people. Consistent with our theory, H-G patients do not get cancer (Martin, 1978).

Telomerase Activity in Epithelial Tissues

Several types of basal epithelial cells (which must proliferate extensively for normal functioning) express telomerase (reviewed in Greider, 1998). Yet basal layers are not a common source of tumors in young people. There are at least two reasons: first, the basal layer is protected from superficial contact with environmental mutagens. Second, progeny of the basal cells are sloughed from the body regularly, likely purging hyper-proliferative cells from these tissues before they become a danger (Cairns, 1975).

III. Conclusions

An Optimal Window of Reproductive Opportunity

Decreasing the rate of human senescence and the threat posed to us by tumors are desirable goals for medicine. Shay and Wright (1999; also see Shay, 1999) have outlined a research plan to accomplish both:
“The key issue is to find out how to make our cancer cells mortal and our healthy cells immortal, or at least longer lasting. Inhibition of telomerase in cancer cells may be a viable target for anti-cancer therapeutics while expression of telomerase in normal cells may extend lifespan.”

This illustrates the danger of isolating medical research from evolutionary biology. If one believes that senescence results from a lack of selection, then they may reasonably pursue a technological solution to fill in where selection left off. But evolutionary theory indicates that senescence results primarily from trade-offs, not from incidental effects or a failure of selection. Once we recognize that longevity and tumor suppression are antagonistic goals, the first question we should seek to answer is “how well has selection optimized the balance between these traits?”

It is not clear that selection has left much room for improvement. We suggest that a staggering majority of our proto-tumor cells are already mortal, allowing only a miniscule risk of tumorigenesis in the first four decades of life. And it is likely that selection has already adjusted our lifespan by modifying telomere lengths and by activating telomerase in early gestation. It is a reasonable guess that maximum longevity cannot be greatly extended without a dramatic increase in the rate of tumor formation, and that increasing the effectiveness of telomeric tumor suppression would accelerate the aging process.

Antagonistic Selection in a Stochastic Environment

Williams (1957) provides a potentially falsifying prediction for his theory: no individual can have an unusually vigorous youth and an unusually long life. We agree no individual should be genetically predisposed to both, but an individual with long telomeres may exhibit slow senescence accompanied by an increased risk of tumor formation yet, by chance, not acquire mutations leading to cancer. It appears that Williams' prediction did not account for the prominent role that environmental stochasticity plays in the senescence equation.

Medical Applications

If a simple modification of telomere-system parameters would extend life without significant costs, selection would surely have found it. We are therefore skeptical of attempts to favorably modify telomere regulation in healthy people. But that does not imply that medical benefits cannot be derived from telomere regulation. In fact, such knowledge holds great medical promise. Telomerase treatment, in vitro, may rejuvenate tissues or organs before transplant, extending telomeres in accordance with the amount of cell division expected to occur in the recipient. This has been suggested for bone marrow transplants (Holt and Shay, 1999) and may be particularly useful for liver transplants in which fractions of a divided liver grow to normal size in multiple recipients.

Further, replacement tissues could be grown from a person's own cells, in the presence of telomerase, to provide a patient threatened by the premature senescence of a tissue with an MHC-matched replacement. This technique might be useful in treating early-stage HIV patients. HIV-reactive T-cells could be removed early in the course of infection, maintained in vitro and treated with telomerase. When the circulating T-cell count begins to crash, the invigorated cells could be reintroduced into the patient where they might greatly extend the HIV latent phase.

Finally, given our increasing ability to detect and surgically eliminate tumors, we may one day be willing to accept an increase in our tumor risk in order to extend youth. The in vitro lengthening of zygote telomeres would likely produce that heritable effect.

Avenues of research likely to lead to viable therapies are those to which natural selection has not had access (e.g. surgery and in vitro methodologies). The idea that medical science will improve the cell-by-cell regulation of telomerase in healthy people, thereby extending youth while at the same time reducing cancer risks, is wishful thinking of the highest order.

Evolutionary Theory: Implications For Current Methodologies

The belief that senescence evolves because the harmful effects of genes are invisible to selection late in life, and thus accumulate by drift, is inadequate to account for the senescence of iteroparous organisms. Despite Williams' (1957) elucidation of this point, a chronic confusion on this issue persists. This oversight has important implications for present and future work, implications which are brought into stark relief by errors in telomere research. A focus on drift as a causal agent has produced misinterpretations of empirical patterns (e.g. senescent cellular phenotypes) and may have obscured others (e.g. developmental coordination of reserve capacities between tissues).

Most importantly, a failure to understand the active way that environmental hazards selectively adjust patterns of senescence has resulted in a haphazard breeding strategy for model organisms like mice. Inadvertent selection has altered our model systems in ways which obscure the very patterns we most wish to understand.

Not only are our model organisms unfit for studies of aging, but because they have extraordinary reserve capacities, their use in the safety testing of drugs, pesticides and other chemical agents is likely to drastically underestimate somatic damage. Toxins which damage tissues, hastening cell line attrition and thereby accelerating organ degeneration, may appear harmless when administered (even in high doses) to mice with telomeres long enough to last six generations. The same substance may produce irreversible effects in humans, which we may fail to recognize if they manifest after a delay of many years and appear similar to normal effects of age. We should therefore reconsider the use of substances deemed safe primarily because they proved harmless to “mice”. At the same time, safety testing with lab mice may have overestimated cancer risks associated with other substances and procedures.

### Literature Cited

Alexander, P. 1966. Is there a relationship between aging, the shortening of life-span by radiation and the induction of somatic mutations? In N. W. Shock (ed.), Perspectives in experimental gerontology, pp. 266-279. Charles C. Thomas, Springfield, Ill.
Alexander, R. D. 1987. The Biology of Moral Systems. A. de Gruyter, Hawthorne, N.Y.
Allsopp, R. C., H. Vaziri, C. Patterson, S. Goldstein, E. V. Younglai, A. B. Futcher, C. W. Greider, and C. B. Harley. 1992. Telomere length predicts replicative capacity of human fibroblasts. Proceedings of the National Academy of Sciences of the United States of America, 89:10114-10118.
Austad, S. N. 1993. Retarded senescence in an insular population of Virginia opossums (Didelphis virginiana). Journal of Zoology, 229:695-708.
Balin, A. K. 1994. Skin changes as reflection of biologic age. In A. K. Balin (ed.), Human Biologic Age Determination, pp. 343-373. CRC Press, Boca Raton.
Black, M. 1969. A modified radiographic method for measuring skin thickness. Br. J. Dermatol., 81:661.
Blackburn, E. H. 1991. Telomeres. Trends in Biochemical Sciences, 16:378-381. 1992. Telomerases. Annual Review of Biochemistry, 61:113-129.
Blasco, M. A., H.-W. Lee, M. P. Hande, E. Samper, P. M. Lansdorp, R. A. Depinho, and C. W. Greider. 1997. Telomere shortening and tumor formation by mouse cells lacking telomerase RNA. Cell, 91:25-34.
Buchkovich, K. J., and C. W. Greider. 1996. Telomerase regulation during entry into the cell cycle in normal human T cells. Molecular Biology of the Cell, 7:1443-1454.
Burnet, F. M. 1957. A modification of Jerne's theory of antibody production using the concept of clonal selection. Austral. J. Sci., 20:67.
Cairns, J. 1975. Mutation selection and the natural history of cancer. Nature, 255:197-200.
Campbell, K. H. S., J. McWhir, W. A. Ritchie, and I. Wilmut. 1996. Sheep cloned by nuclear transfer from a cultured cell line. Nature, 380:64-66.
Campisi, J. 1997. Aging and cancer: The double-edged sword of replicative senescence. Journal of the American Geriatrics Society, 45:482-488. 1997. The biology of replicative senescence. European Journal of Cancer, 33:703-709.
Campisi, J., G. Dimri, and E. Hara. 1996. Control of replicative senescence. In E. L. Schneider and J. W. Rowe (eds.), Handbook of the Biology of Aging, pp. 121-149. Academic Press, San Diego.
Chang, E., and B. Harley Calvin. 1995. Telomere length and replicative aging in human vascular tissues. Proceedings of the National Academy of Sciences of the United States of America, 92:11190-11194.
Chiu, C.-P., and B. Harley Calvin. 1997. Replicative senescence and cell immortality: The role of telomeres and telomerase. Proceedings of the Society for Experimental Biology & Medicine, 214:99-106.
Counter, C. M., H. W. Hirte, S. Bacchetti, and C. B. Harley. 1994. Telomerase activity in human ovarian carcinoma. Proceedings of the National Academy of Sciences of the United States of America, 91:2900-2904.
de Lange, T. 1998. Telomeres and senescence: Ending the debate. Science, 279:334-335.
Eriksson, J. G., T. Forsen, J. Tuomilehto, P. D. Winter, C. Osmond, and D. J. P. Barker. 1999. Catch-up growth in childhood and death from coronary heart disease: Longitudinal study. British Medical Journal, 318:427-431.
Faragher, R. G. A., I. R. Kill, J. A. A. Hunter, F. M. Pope, C. Tannock, and S. Shall. 1993. The gene responsible for Wemer syndrome may be a cell division “counting” gene. Proceedings of the National Academy of Sciences of the United States of America, 90:12030-12034.
Feng, J., W. D. Funk, S.-S. Wang, S. L. Weinrich, A. A. Avilion, C.-P. Chiu, R. R. Adams, E. Chang, R. C. Allsopp, J. Yu, S. Le, M. D. West, C. B. Harley, W. H. Andrews, C. W. Greider, and B. Villeponteau. 1995. The RNA component of human telomerase. Science, 269:1236-1241.
Forsen, T., J. G. Eriksson, J. Tuomilehto, C. Osmond, and D. J. P. Barker. 1999. Growth in utero and during childhood among women who develop coronary heart disease: Longitudinal study. British Medical Journal, 319:1403-1407.
Greider, C. W. 1998. Telomerase activity, cell proliferation, and cancer. Proceedings of the National Academy of Sciences of the United States of America, 95:90-92. 1999. Telomeres do d-loop-t-loop. Cell, 97:419-422.
Griffith, J. K., J. E. Bryant, C. A. Fordyce, F. D. Gilliland, N. E. Joste, and R. K. Moyzis. 1999. Reduced telomere DNA content is correlated with genomic instability and metastasis in invasive human breast carcinoma. Breast Cancer Research & Treatment, 54:59-64.
Harley, C. B. 1997. Human ageing and telomeres. In D. J. Chadwick and G. Cardew (eds.), Telomeres and Telomerase, Vol. 211, pp. 129-144. John Wiley & Sons, Chichester.
Harley, C. B., A. B. Futcher, and C. W. Greider. 1990. Telomeres shorten during aging of human fibroblasts. Nature, 345:458-460.
Hastie, N. D., M. Dernpster, M. G. Dunlop, A. M. Thompson, D. K. Green, and R. C. Allshire. 1990. Telomere reduction in human colorectal-carcinoma and with aging. Nature, 346:866-868.
Hayflick, L. 1973. Biology of human aging. American Journal of the Medical Sciences, 265:432-445. 1991. Aging under glass. Mutation Research, 256:69-80.
Hayflick, L., and P. S. Moorhead. 1961. The serial cultivation of human diploid cell strains. Exp. Cell Res., 25:585-621.
Holt, S. E., and J. W. Shay. 1999. Role of telomerase in cellular proliferation and cancer. Journal of Cellular Physiology, 180:10-18.
Horwich, A. 1996. Testicular germ cell tumors: An introductory overview. In A. Horwich (ed.), Testicular cancer: Investigation and management, pp. 1-17. Chapman & Hall Medical, London.
Jennings, B. J., S. E. Ozanne, M. W. Dorling, and C. N. Hales. 1999. Early growth determines longevity in male rats and may be related to telomere shortening in the kidney. Febs Letters, 448:4-8.
Johnson, F. B., D. A. Sinclair, and L. Guarente. 1999. Molecular biology of aging. Cell, 96:291-302.
Khalifa, M. M. 1989. Hutchinson-Gilford Progeria Syndrome Report of a Libyan Family and Evidence of Autosomal Recessive Inheritance. Clinical Genetics, 35:125-132.
Kim, N. W., M. A. Piatyszek, K. R. Prowse, C. B. Harley, M. D. West, P. L. C. Ho, G. M. Coviello, W. E. Wright, S. L. Weinrich, and J. W. Shay. 1994. Specific association of human telomerase activity with immortal cells and cancer. Science, 266:2011-2015.
Kipling, D. 1995. The Telomere. Oxford University Press, Oxford. 1997. Mammalian telomerase: Catalytic subunit and knockout mice. Human Molecular Genetics, 6:1999-2004.
Kipling, D., and H. J. Cooke. 1990. Hypervariable ultra-long telomeres in mice. Nature, 347:400-402.
Lanza, R. P., J. B. Cibell, C. Blackwell, V. J. Cristofalo, M. K. Francis, G. M. Baerlocher, J. Mak, M. Schertzer, E. A. Chavez, N. Sawyer, P. M. Lansdorp, and M. D. West. 2000. Extension of cell life-span and telomere length in animals cloned from senescent cells. Science, 288:665-668.
Lee, H. W., M. A. Blasco, G. J. Gottlieb, J. W. Horner, C. W. Greider, and R. A. DePinho. 1998. Essential role of mouse telomerase in highly proliferative organs. Nature, 392:569-574.
Li, Y., B. Deeb, W. Pendergrass, and N. Wolf. 1996. Cellular proliferative capacity and life span in small and large dogs. Journal of Gerontology, 51A:B403-B408.
Lindsey, J., N. I. McGill, L. A. Lindsey, D. K. Green, and H. J. Cooke. 1991. In-vivo loss of telomeric repeats with age in humans. Mutation Research, 256:45-48.
Ly, D. H., D. J. Lockhart, R. A. Lemer, and P. G. Schultz. 2000. Mitotic misregulation and human aging. Science, 287:2486-2492.
Maciel, A. T. 1988. Evidence For Autosomal Recessive Inheritance of Progeria Hutchinson Gilford. American Journal of Medical Genetics, 31:483-488.
Martin, G. M. 1978. Genetic syndromes in man with potential relevance to the pathobiology of aging. In D. Bergsma and D. E. Harrison (eds.), Genetic effects on aging, pp. 5-39. Alen R. Liss, New York.
Medawar, P. B. 1952. An Unsolved Problem in Biology. H. K. Lewis, London.
Michell, A. R. 1999. Longevity of British breeds of dog and its relationships with sex, size, cardiovascular variables and disease. Veterinary Record, 145:625-629.
Moyzis, R. K., J. M. Buckingham, L. S. Cram, M. Dani, L. L. Deaven, M. D. Jones, J. Meyne, R. L. Ratliff, and J. R. Wu. 1988. A highly conserved repetitive DNA-sequence, (TTAGGG), present at the telomeres of human chromosomes. Proceedings of the National Academy of Sciences of the United States of America, 85:6622-6626.
National Research Council. 1981. Mammalian models for research on aging. National Academy Press, Washington, D.C.
Olovnikov, A. M. 1973. A theory of marginotomy: The incomplete copying of template margin in enzymic synthesis of polynucleotides and biological significance of the phenomenon. Journal of Theoretical Biology, 41:181-190.
Profet, M. 1988. The evolution of pregnancy sickness as protection to the embryo against pleisiocene teratogens. Evolutionary Theory, 8:177-190.
Reznick David, N. 1997. Life history evolution in guppies (Poecilia reticulata): Guppies as a model for studying the evolutionary biology of aging. Experimental Gerontology, 32:245-258.
Ricklefs, R. E. 1998. Evolutionary theories of aging: Confirmation of a fundamental prediction, with implications for the genetic basis and evolution of life span. American Naturalist, 152:24-44.
Rohme, D. 1981. Evidence for a relationship between longevity of mammalian species and life spans of normal fibroblasts in vitro and erythrocytes in vivo. Proceedings of the National Academy of Sciences of the United States of America—Biological Sciences, 78:5009-5013.
Ross, M. H. 1976. Nutrition and longevity in experimental animals. In M. Winick (ed.), Nutrition and aging. John Wiley & Sons, New York.
Rudolph, K. L., S. Chang, H.-W. Lee, M. Blasco, G. J. Gottlieb, C. Greider, and R. A. DePinho. 1999. Longevity, stress response, and cancer in aging telomerase-deficient mice. Cell, 96:701-712.
Samaras,T. T., and H. Elrick. 1999. Height, body size and longevity. Acta Medica Okayama, 53:149-169.
Samaras, T. T., and L. H. Storms. 1992. Impact of height and weight on life-span. Bulletin of the World Health Organization, 70:259-267.
Shay, J. W. 1999. Toward identifying a cellular determinant of telomerase repression. Journal of the National Cancer Institute, 91:4-6.
Shay, J. W., and W. E. Wright. 1999. Telomeres and telomerase in the regulation of human cellular aging. In V. A. Bohr, B. F. C. Clark and T. Stevnsner (eds.), Molecular Biology of Aging. Alfred Benzon symposium 44, pp. 148-158, Munksgaard, Copenhagen.
Shiels, P. G., A. J. Kind, K. H. S. Campbell, D. Waddington, I. Wilmut, A. Colman, and A. E. Schnieke. 1999. Analysis of telomere lengths in cloned sheep. Nature, 399:316-317.
Smith, J. R., and 0. M. Pereira-Smith. 1996. Replicative senescence: Implications for in vivo aging and tumor suppression. Science, 273:63-67.
Sprott, R. L., and S. N. Austad. 1996. Animal models for aging research. In E. L. Schneider and J. W. Rowe (eds.), Handbook of the biology of aging, pp. 3-23. Academic Press, San Diego.
Stabile, I., G. Grudzinskas, and T. Chard. 1992. Spontaneous Abortion, pp. 231. Springer-Verlag, London.
Thompson, D. a. W. 1943. On Growth and Form. 2nd ed. Cambridge University Press, Cambridge.
Ulaner, G. A., and L. C. Giudice. 1997. Developmental regulation of telomerase activity in human fetal tissues during gestation. Molecular Human Reproduction, 3:769-773.
Watson, J. 1972. Origin of concatameric T7 DNA. Nat. N. Biol., 239:197-201.
Weinrich, S., L., R. Pruzan, L. Ma, M. Ouellette, V. M. Tesmer, S. E. Holt, A. G. Bodnar, S. Lichsteiner, N. W. Kim, J. B. Trager, R. D. Taylor, R. Carlos, W. H. Andrews, W. E. Wright, J. W. Shay, C. B. Harley, and G. B. Morin. 1997. Reconstitution of human telomerase with the template RNA component hTR and the catalytic protein subunit hTRT. Nature Genetics, 17:498-502.
Weng, N. P., L. Granger, and R. J. Hodes. 1997. Telomere lengthening and telomerase activation during human B cell differentiation. Proc. Natl. Acad. Sci. U.S.A., 94:10827-10832.
Williams, G. C. 1957. Pleiotropy, natural selection, and the evolution of senescence. Evolution, 11:398-411.
Wolf, N. S., P. E. Penn, D. Z. Jiang, R. G. Fei, and W. R. Pendergrass. 1995. Caloric restriction: Conservation of in vivo cellular replicative capacity accompanies life-span extension in mice. Experimental Cell Research, 217:317-323.
Wright, W. E., M. A. Piatyszek, W. E. Rainey, W. Byrd, and J. W. Shay. 1996. Telomerase activity in human germline and embryonic tissues and cells. Developmental Genetics, 18:173-179.
Yashima, K., A. Maitra, B. B. Rogers, C. F. Timmons, A. Rathi, H. Pinar, W. E. Wright, J. W. Shay, and A. F. Gazdar. 1998. Expression of the RNA component of telomerase during human development and differentiation. Cell Growth & Differentiation, 9:805-813.

1. A method of breeding an animal line for experimental use, comprising:
(a) preselecting a first population comprising a plurality of conspecific animals of breeding age;
(b) preselecting a plurality of breeding periods of varying lengths;
(c) arbitrarily assigning one of the preselected breeding periods to each animal of the first population; and
(d) allowing each animal of the first population to breed only during its assigned breeding period.

2. The method of claim 1, wherein arbitrarily assigning a breeding period in step (c) is accomplished by a random method or a pseudo-random method.

3. The method of claim 1, wherein the preselected breeding periods are each bounded by a first chronological age and an older second chronological age of the animal.

4. The method of claim 1, wherein the preselected breeding periods are each bounded by a first calendar date and a later second calendar date.

5. The method of claim 1, wherein at least one of the preselected breeding periods assigned to an animal of the first population has no retirement date.

6. The method of claim 5, wherein a plurality of breeding periods assigned to animals of the first population have no retirement date.

7. The method of claim 1, wherein the animal is a member of an order selected from the group consisting of Carnivora, Primates, Rodentia, Insectivora, and Lagomorpha.

8. The method of claim 7, wherein the animal is a member the order Rodentia.

9. The method of claim 8, wherein the animal is a mouse.

10. The method of claim 9, wherein at least one preselected breeding period is longer than 8 months.
